(19) United States
(12) Patent Application Publication
(10) Pub. No.: US 2021/0116488 A1
(43) Pub. Date: Apr. 22, 2021

(54) Quantum Converting Nanoparticles as In Vivo and In Situ Optical Electric Field Sensors

(71) Applicant: The Board of Trustees of the Leland Stanford Junior University, Stanford, CA (US)

(72) Inventors: Randy Mehlenbacher, Palo Alto, CA (US); Alice Lay, Palo Alto, CA (US); Rea Kolbl, Boulder, CO (US); Miriam Goodman, Menlo Park, CA (US); Jennifer Dionne, Menlo Park, CA (US)

(21) Appl. No.: 17/109,010

(22) Filed: Dec. 1, 2020

Related U.S. Application Data
(63) Continuation of application No. 16/381,874, filed on Apr. 11, 2019.
Application No. 16/381,874 is a continuation-in-part of application No. 16/191,901, filed on Nov. 15, 2018, now abandoned.
(60) Provisional application No. 62/586,776, filed on Nov. 15, 2017.

(51) Int. Cl. G01R 29/08; A61K 49/00

(52) U.S. Cl. CPC G01R 29/0885; A61K 49/0067

(57) ABSTRACT

Quantum converting nanoparticles for electric field sensing are provided. In one example, by combining upconverting lanthanide ions with voltage responsive dyes, we generate an optical platform that displays intensity and spectrum changes in the presence of electric fields. Our particles enable local (down to 10 nm spatial resolution) mapping of electric fields with exceptional photostability. We can image and quantify in vivo and in situ electric fields in biological and material systems up to fields of ˜100 kV/cm.

### CROSS REFERENCE TO RELATED APPLICATIONS

This application is a continuation of U.S. patent application Ser. No. 16/381,874, filed on Apr. 11, 2019 and hereby incorporated by reference in its entirety.

Application Ser. No. 16/381,874 is a continuation in part of U.S. patent application Ser. No. 16/191,901, filed on Nov. 15, 2018 and hereby incorporated by reference in its entirety.

Application Ser. No. 16/191,901 claims the benefit of U.S. provisional patent application 62/586,776, filed on Nov. 15, 2017, and hereby incorporated by reference in its entirety.

### FIELD OF THE INVENTION

This invention relates to quantum converting nanoparticles for electric field detection.

### BACKGROUND

Electric fields are used by most living organisms for communication and interpretation of information within the brain and muscles. Moreover, electric fields are prevalent in many of the devices we use every day from our phones to computers. Local electric fields are difficult to characterize and study in vivo and in situ. Accordingly, it would be an advance in the art to provide improved electric field measurements.

### SUMMARY

Here we introduce an all optical approach that leverages quantum converting nanoparticles for in vivo and in situ investigation of electric fields. In one example, by combining upconverting lanthanide ions with voltage responsive dyes, we generate an optical platform that displays intensity and spectrum changes in the presence of electric fields. Our particles enable local (down to 10 nm spatial resolution) mapping of electric fields with exceptional photostability. We can image and quantify in vivo and in situ electric fields in biological and material systems up to fields of ˜100 kV/cm.

Our work demonstrates electric field sensitive upconversion in a nanoparticle platform. Upconverting electric field sensors have applications in many fields. In the field of electrophysiology, this technology offers probing electric fields of biological relevance (<10 nm, up to 100 kV/cm) in living organisms. Applications include characterizing cardiomyocyte activity, studying neural networks, optical imaging of action potential and brain activity. Other biological applications include studying electric fields used by animals for direction and prey sensing. Beyond, these nanoparticle field sensors could provide local understanding of dielectric breakdown in a variety of devices and materials. Further, these particles may be used to understand electric field inhomogeneities in devices that use electric fields such as printers, electric motors, batteries, etc.

Significant advantages are provided by this work. To date, most electric field sensors are limited by poor signal to noise ratio (cannot detect single electric field spikes) and photodegradation (photoblinking and/or photobleaching) hindering long-term measurements. Although some inorganic sensors may have an electric field response, the stimuli response is weak and these platforms can be toxic. In the areas of monitoring equipment, there are limited technologies currently available to characterize electric fields over time. Existing sensors are generally mounted on the exterior of the equipment or at an interface. Upconverting electric field sensors embedded in the material matrix itself would provide valuable electric field maps, with high spatial resolution, in order to locate field inhomogeneities.

This nanoparticle platform for electric field detection is the first hybrid organic-inorganic (or inorganic) platform to use both intensity and spectro-ratiometric (relative color change) readout for the determination of local electric fields in vitro, in vivo, and in situ.

### BRIEF DESCRIPTION OF THE DRAWINGS

FIGS. 1A-B schematically show operation of two embodiments of the invention.

FIG. 2A shows the energy level scheme for the experiments of section B.

FIG. 2B shows dye emission and absorption spectra compared with Er³⁺ emission spectra.

FIG. 2C schematically shows core-shell nanoparticles as used in this work.

FIG. 2D is an image of as-fabricated nanoparticles prior to polymer wrapping.

FIG. 3A shows upconversion and photoluminescence spectra for core-shell nanoparticles.

FIGS. 3B-D show voltage-dependent results for the upconverting nano-particles of FIG. 3A.

FIG. 4 shows upconversion spectra for nanoparticles with and without dye.

FIG. 5 shows upconversion spectra for dye-coated nanoparticles with and without an electric field.

FIG. 6 shows cycling results for the dye-coated nanoparticles of FIG. 5.

### DETAILED DESCRIPTION

### A) General Principles

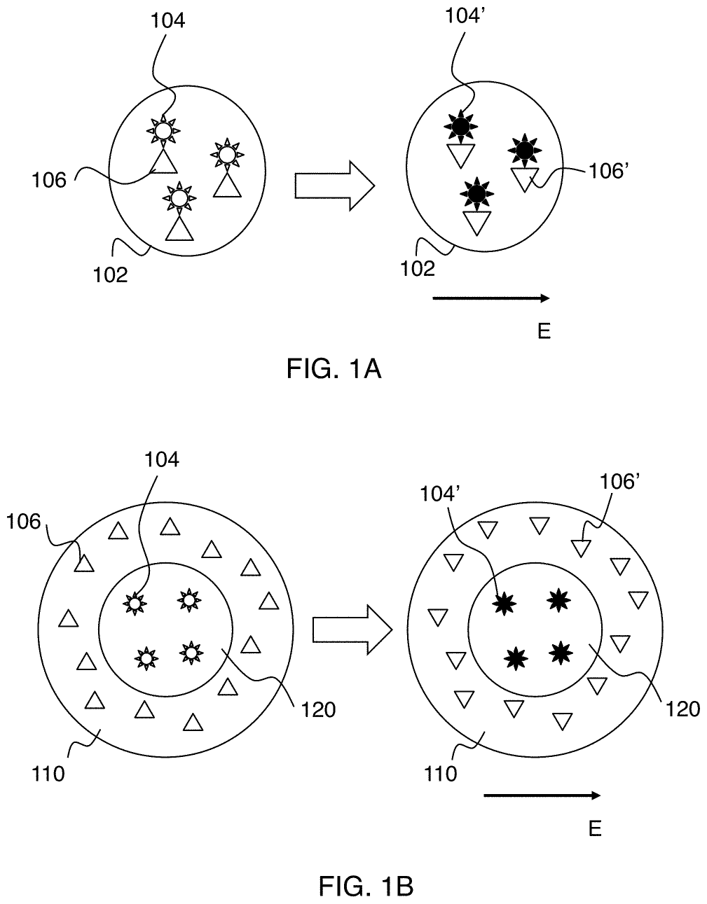

The approach of this work is to decorate existing quantum converting nanoparticles (e.g. NaYF₄ host lattices doped with Yb³⁺ and Er³⁺) with voltage sensitive dyes (DI-2-ANEPEQ for example). The electronic states of the quantum converting nanoparticles will couple with the voltage sensitive dye. Because the electronic states of the dye are modulated by an external field, the coupling will change in the presence of an electric field and thus the spectrum and intensity of light emission will change. FIG. 1A schematically shows this embodiment, where nano-particle matrix 102 includes lanthanide dopant(s) 104 that are coupled to voltage sensitive dye(s) 106. Application of an electric field causes the dye to change its configuration, as schematically shown by the different orientations of 106 and 106′. This change alters the emission from the lanthanide dopant(s), as schematically shown by dopant(s) 104′ being shaded differently than dopant(s) 104.

FIG. 1B shows a presently preferred embodiment, where the nanoparticle has a core 120 surrounded by a shell 110, where the relevant dopants 104, 104′ are in core 120 and where the voltage sensitive dye 106, 106′ is in shell 110. Here a nanoparticle is a particle having a largest dimension of one micron or less. At the wavelengths of this work, nano-scale spatial separation between the lanthanide dopants and the voltage sensitive dye, as shown, does not substantially reduce their mutual coupling.

Accordingly, one embodiment of the invention is an apparatus for sensing electric fields. The apparatus includes one or more nanoparticles configured to receive incident radiation at a first wavelength and to provide output radiation at a second wavelength via quantum conversion, where the first and second wavelengths are distinct. The apparatus also includes one or more dyes having a quantum Stark response to an electric field. The dyes are disposed on or in the nanoparticles such that one or more parameters of the quantum conversion are altered by the electric field due to coupling between the dyes and the nanoparticles.

As in the example of FIG. 1B, the nanoparticles can be configured as a core covered by a shell, where the dyes are preferentially disposed in the shell. The shell can be formed by wrapping a polymer around the core.

The parameters of the quantum conversion that can change due to an applied electric field include, but are not limited to: intensity at an output wavelength, and/or spectral intensity in an output wavelength range. Appropriate calibration of these changes will enable quantitative measurements of local electric fields.

The nanoparticles can be configured to be disposed on or in a biological specimen to provide in vitro electric field sensing with sub-micron spatial resolution. The nanoparticles can be configured to be disposed on or in a living biological subject to provide in vivo electric field sensing with sub-micron spatial resolution. The nanoparticles can be configured to be disposed on or in an electrical device or machine to provide in situ electric field sensing with sub-micron spatial resolution.

The nanoparticles can include one or more lanthanide converting materials having doping selected from the group consisting of: Er doping, Yb doping, Tm doping, Nd doping and mixtures or combinations thereof.

The one or more dyes can be selected from the group consisting of: aminonaphthylethenylpyridinium dyes and photoinduced electron transfer dyes. Here aminonaphthylethenylpyridinium dyes are any dyes obtained from aminonaphthylethenylpyridinium by substitution of other organic groups for one or more of its hydrogen atoms. Photoinduced electron transfer dyes are any dyes having electron transfer as its mechanism for providing voltage sensitivity.

Although the examples herein all relate to the case of upconversion (i.e., second wavelength shorter than the first wavelength), downconversion (i.e., second wavelength longer than the first wavelength) is also possible. For biological applications of upconverting embodiments, it is preferred for the first wavelength to be in a range from 0.74 μm to 2.0 μm (i.e., near-IR) and for the second wavelength to be in a range from 380 nm to 740 nm (i.e., visible). Alternatively, the first and second wavelengths can both be in the 0.74 to 2.0 μm range. Lanthanide-based upconversion offers many advantages for imaging and sensing, because it absorbs near infrared photons and emits in the visible spectrum. In biology, this yields minimal tissue autofluorescence and deeper tissue penetration. These nanoparticles can be incorporated into the system either through natural uptake (e.g. digestion or absorption) or artificial introduction (e.g. injection, electroporation).

Several variations are possible. The nanoparticle dopant concentrations is one possible tuning parameter accessible to these nanosensors. By using different lanthanide ions, different color sensors can be designed to limit the nanoparticle optical interaction with the sample. A core-shell morphology may enhance the lanthanide/voltage sensitive dye coupling enhancing the electric field sensitivity. Overall, the nanoparticle platform opens a variety of possibilities in surface functionalization for targeting specific cellular or material regions of interest.

### B) EXPERIMENTAL EXAMPLES

### B1 Introduction

Electric fields abound in our daily lives. In biology, changes in electric fields are used to transmit information in the form of action potentials and some animals, such as eels and bees, use electric fields for tracking, hunting, and protection. In meteorology, variations in electric fields can be used to assist in weather prediction. In semiconductor technology, local electric fields dictate breakdown thresholds in photovoltaics and the function of a variety of organic electronics, including polymer transistors and diodes. Safety in process control engineering relies critically on accurate detection and quantification of electric fields.

Despite the pervasive nature of electric fields, observation of local electric fields is challenging. Invasive, complex electrical arrays can be wired and placed in the vicinity of the system of interest, but these arrays have to balance electrode density, electrode shielding complexity, and electrode invasiveness. Optical electric field sensors, in contrast, can be designed such that they have limited system perturbation, sub-micron spatial resolution, fast response times, and no cross-talk. These optical sensors exhibit a change in intensity or wavelength with an applied electric field, and a wide array of optical electric field sensors have been developed including quantum dots, nitrogen-vacancies in nanodiamonds, genetically encoded voltage indicators (GEVIs), and voltage sensitive dyes (VSDs).

These probes, however, are limited by their ultraviolet (UV) or visible excitation, which leads to limited material penetration depth and generally large optical backgrounds due to material autofluorescence. Additionally, several of these probes (i.e. GEVIs and VSDs), have been developed largely for imaging action potentials and are not readily translated to applications outside of biology.

### B2) Approach

To overcome these materials limitations, we have developed a new electric field sensor, based on coupling upconverting nanoparticles (UCNPs) to VSDs. UCNPs are a promising class of fluorophores that absorb near-infrared (NIR) light and emit visible photons. Because this nanostructure composite absorbs lower energy photons than it emits, autofluorescence from the surrounding media does not interfere with the signal allowing for background-free imaging. Furthermore, because of the NIR excitation, deeper penetration depths can be achieved at fluences far lower than those used in traditional two photon probes. By selecting different lanthanide ions, doping concentrations, and dyes, different emission wavelengths can be realized.

We design our UCNPs to exhibit visible emission that is strongly coupled to the absorption of the VSD. The VSD provides the composite inorganic-organic system with an electric field response that UCNPs do not intrinsically possess. To enhance coupling to and emission from the VSDs in this example, we wrap the UCNPs with a poly(maleic anhydride-alt-1-octadecene) (PMAO) polymeric shell. This polymer-wrapping also enables transfer to either polar or nonpolar media, enabling integration into a wide range of materials and environments. Our composite system combines the best properties from each component, namely NIR excitation and visible emission from the nanoparticles with the fast, nanosecond response time and high electric field sensitivity of the VSDs.

### B3) Results and Discussion

We utilize a β-NaLnF₄ host lattice for our upconverting nanoparticles (UCNPs) as this host lattice enables the largest upconversion efficiency to date. The β-NaLnF₄ lattice is not spontaneously polarizable, so these nanoparticles do not have an intrinsic response to an external electric field and therefore cannot, on their own, report the local electric field of a system. We hypothesized that dye-decoration of the UCNP surface could provide such field sensitivity. Many reports have coupled dye molecules to UCNPs to enhance the nanoparticles' absorption and emission properties. These molecules are selected such that their absorptive states match either the nanoparticle absorption or emission wavelength.

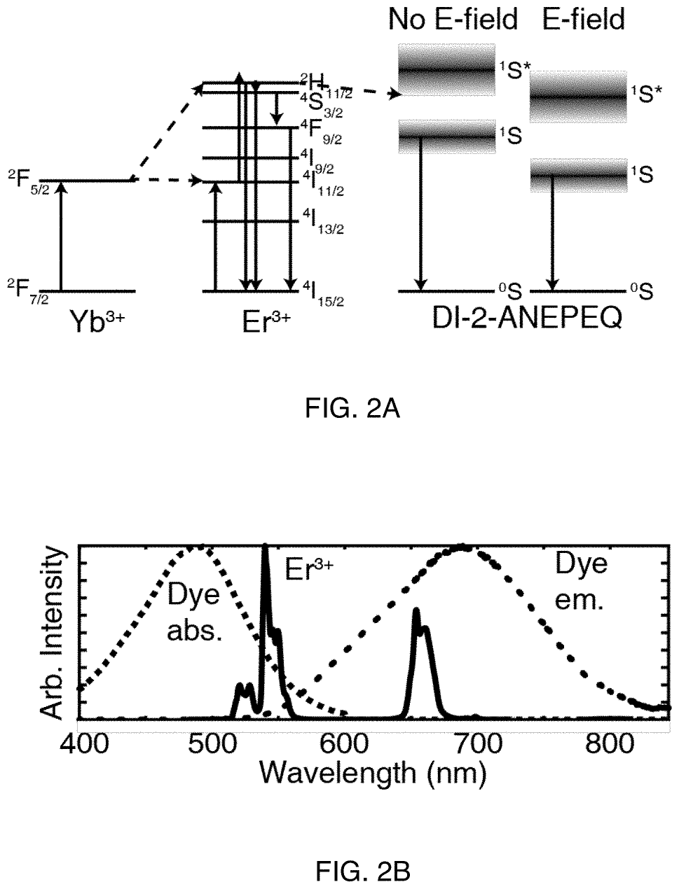

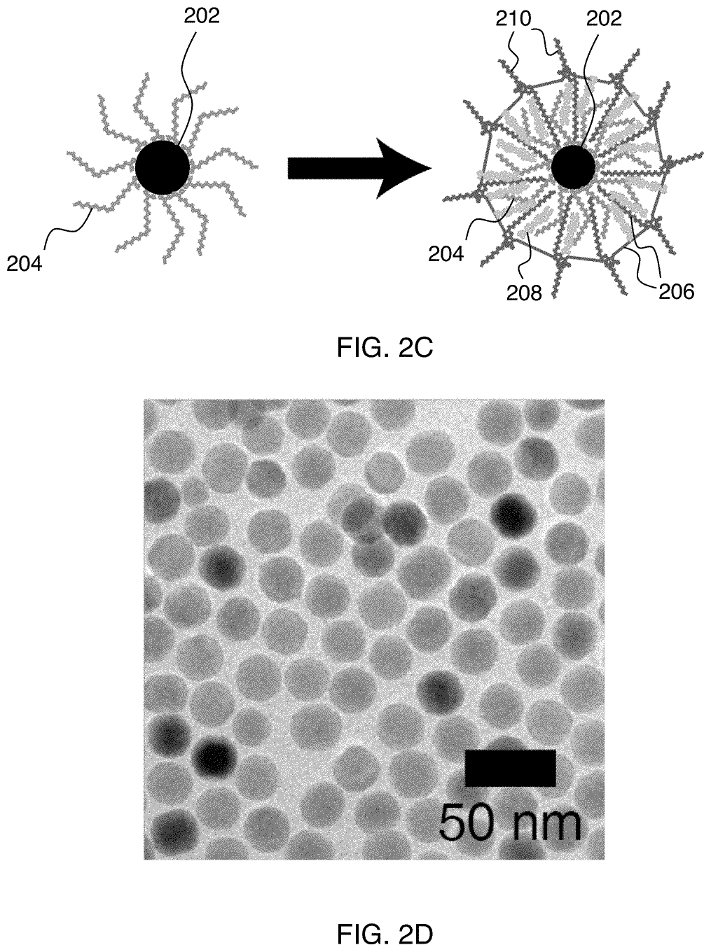

To create a composite UCNP-dye system that has an optical response to electric fields, we select a dye molecule which undergoes a large Stark shift in the presence of an electric field. As with passive dye-UCNP systems, the VSD needs to spectrally match the upconverter's energy levels (see FIG. 2A). When an electric field is present, both the absorptive and emissive energy levels of the dye shift. This change in energy levels leads to a change in the coupling between the upconverters and the dye affecting both the intensity and color of emission from the system.

Here, we chose the voltage sensitive dye DI-2-ANEPEQ (Pyridinium, 4-[2-[6-(diethylamino)-2-naphthalenyl]ethenyl]-1-[2-(trimethylammonio)ethyl]-, dibromide) as it shows the best optical overlap with the Er³⁺ emissive states. FIG. 2B shows the absorption and emission spectra for DI-2-ANEPEQ dissolved in water compared to the Er³⁺ emission spectrum. The ²H_{11/2} and ⁴S^{3/2} states in Er³⁺ ions, at ˜525 and ˜540 nm, are well matched to the absorption in DI-2-ANEPEQ; thus these Er³⁺ states will transfer energy to dye molecules near the UCNP surface. Using the overlap between the Er³⁺ emission and DI-2-ANEPEQ absorption spectra, we estimate that the Forster radius between Er³⁺ and this dye is 8.2 nm.

We synthesize β-NaY_{0.8}Y_{0.8}Er_{0.02}F₄ nanoparticles following well-established colloidal synthesis procedures. A transmission electron microscope (TEM) image of these nanoparticles is shown in FIG. 2D. The nanoparticles exhibit a spherical morphology and good size monodispersity of 24±2 nm. For this average nanoparticle diameter, the FRET efficiency is estimated to be at least 0.1.

Unlike other dye molecules that have been coupled to UCNPs, VSDs typically do not have functional groups that can bond to the nanoparticle surface, making dye coupling challenging. Moreover, solvent-exposed VSD molecules have low QY (quantum yield) in contrast to VSD embedded in membranes which minimizes the background fluorescence from unbound dye while imaging. To overcome these issues and prepare a modular system that can be soluble in polar or nonpolar solvents, we have adopted a polymer wrapping scheme for the nanoparticles, shown in FIG. 2C.

First, polymaleic anhydride alt-1-octadecene (PMAO) 206 is wrapped around the nanoparticles 202. The octadecene moieties in the polymer intercalate within the oleic acid ligand shell 204 surrounding the as-synthesized nanoparticles and leave the maleic acid moieties exposed to the surface. Reaction between the maleic anhydride moieties and primary amines of poly(ethylene glycol) (PEG-NH₂) 210 renders the nanoparticles water-soluble; we note that solubility in organic solvents can be readily achieved by choosing different primary amines. This maleic anhydride-amine reaction leaves the nanoparticle surface negatively charged, while DI-2-ANEPEQ has a positive charge. Thus, analogous to the situation in cell membranes, dye molecules 208 are electrostatically driven to intercalate within the oleate/PMAO bilayer and orient the electrostatic charges. By observing dye photoluminescence (PL) under UV lamp excitation, we see greatly enhanced emission from samples that contain polymer micelles, compared to the same concentration of freely dissolved dye. This enhancement in the PL suggests that dye molecules are indeed intercalated within the ligand bilayer.

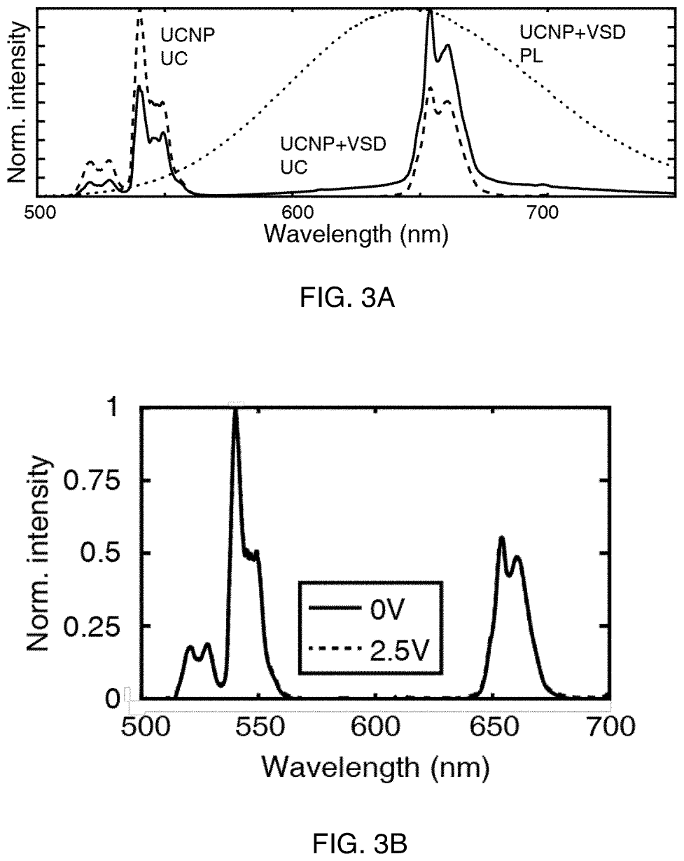

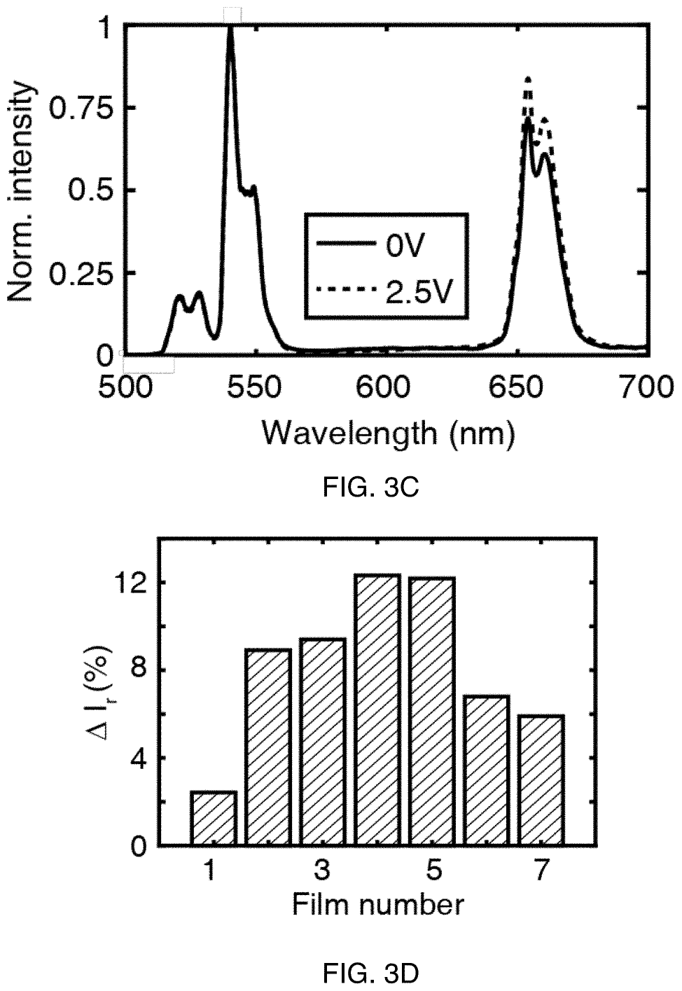

Having successfully bound the dye molecules to the nanoparticle, we measure the upconversion spectra for a dropcast film including the composite UCNP-VSD system. Under NIR illumination at a wavelength of 980 nm, the upconverting nanoparticle film without dye appears green in an optical image. Its spectrum is predominantly comprised of the ²H_{11/2} (525 nm), ⁴S_{3/2} (540 nm), and ⁴F_{9/2} (650 nm) transitions within the Er³⁺ 4f manifold of electronic states (FIG. 3A). The solid line on FIG. 3A is the upconversion spectrum from UCNP+VSD, the dashed line on FIG. 3A is the upconversion spectrum from UCNP without dye, and the dotted line on FIG. 3A is the photoluminescence (PL) spectrum from UCNP+VSD. When DI-2-ANEPEQ molecules are included, the relative intensities of the Er³⁺ peaks changes, increasing the 650 nm emission and decreasing the 525 and 540 nm emission, leading to a redder appearance in upconversion. In addition, a new, broad peak emerges centered around 665 nm. This new peak corresponds to direct emission from the dye molecule itself. Thus, the upconverting nanoparticle can optically excite the dye molecules.

With the confirmation of successful optical coupling between the dye and UCNPs, we apply an electric field to films of these materials. The relative permittivity of the PMAO coating on the nanoparticles is expected to be large; εᵣ for maleic anhydride is ˜50 and for PEG is ˜80; therefore, the electric field experienced by the nanoparticle will be heavily screened from external fields. To overcome this issue, we use an ionic liquid to apply an electric field. In analogy to a neural membrane, with an applied voltage, ions from the ionic liquid penetrate the film leading to large local electric fields as the ions are distributed near the polymer coating surface. To perform these measurements, we dropcast a film of nanoparticles on an ITO (indium tin oxide) glass substrate. A droplet of ionic liquid, 1-Ethyl-3-methylimidazolium bis(trifluoromethylsulfonyl)imide ([EMIM] [TFSI]), is placed on top of the film and a positive voltage is applied while the ITO is held at ground. Thus, a capacitor sustaining a large electric field is prepared across the nanoparticle film, driving ions into the polymer matrix.

To estimate the film thickness and place a lower bound on the electric field strength, we use a focused ion beam (FIB) to mill into the polymer film and then collect a cross-sectional SEM image. We find that the average film thickness is ˜400 nm and that the nanoparticles are well-dispersed throughout the polymer film. Accounting for the electrochemical potential window of [EMIM] [TFSI] (−2 V to 2.5 V depending on the reference electrode), we limit the voltages we apply to avoid oxidizing or reducing the electrolyte. Taken together, the lower bound for electric fields applied across this composite material is −1 kV/cm assuming εᵣ˜65 (65 kV/cm assuming εᵣ=1), ion intercalation within the polymer matrix likely facilitates higher field build-up. For reference, the electric fields present are ˜100 kV/cm (assuming no dielectric screening) during action potential spiking, 0.5 kV/cm in a thundercloud, and 20 kV/cm in light emitting electrochemical cells. Thus, this device is capable of generating electric fields comparable to those of a wide variety of phenomena.

FIGS. 3B-C show the results of the upconversion spectra on a film of nanoparticles with DI-2-ANEPEQ in an applied electric field, with an applied voltage of 2.5 V. Here FIG. 3B is UCNPs with no dye and FIG. 3C is UCNP+dye as described above. We observe an increase in the emission from the red 640 nm Er³⁺ peak relative to the green 525 and 540 nm peaks. The mechanism for this change in red emission comes from 1) shifting of the dye's electronic states, enhancing the dye's absorption of the 525 and 540 nm light 2) more dye molecules contributing to emission and 3) energy back transfer from the dye into the Er³⁺ 640 nm state. The degree of spectral response correlates with the strength of the local electric field. The increase in the red emission intensity when normalizing the green emission (ΔIᵣ) varies for different films from 2.5% to 12%, with a mean response of 9.5% across 7 films (FIG. 3D). This variance arises from differences in film thickness and the relative dye to UCNP ratio between films. This variance matches the normal specification for the intensity fluctuations of as purchased DI-2-ANEPEQ (1-10%), therefore this composite retains similar if not better electric field sensitivity to that of its components.

As a control experiment, we measured the extent of dye photobleaching to confirm that the electric field measurements are not due to dye degradation. Over 30 minutes, we observe a modest ˜10% decrease in signal intensity from the VSD. We find that with photobleaching, the upconversion spectra exhibits reduced red emission relative to the green emission. This enhanced green emission is expected as the dye molecules degrade and less energy is syphoned from the Er³⁺ states. Accordingly, the increased red response we observe with an applied electric field is not due to VSD photobleaching. To verify stability of our capacitive structure, we measured cyclic voltammograms (CV) for the composite system using a silver/silver triflate (Ag/AgOTf) ionic liquid reference electrode. These measurements confirm that no redox reactions occur at our working electrode composite film at applied potentials and we can cycle the electric fields these composite systems experience (at varying scan rates) without degrading the material.

### B4) Conclusion

In conclusion, we have demonstrated the use of a composite organic-inorganic system of UCNPs optically coupled to voltage sensitive dyes. NIR optically excited nanoparticles transfer energy to dye molecules whose electronic states are sensitive to local electric fields; therefore, the overall spectral response of the composite changes in the presence of an electric field. We observe a relative increase of the red upconversion emission ranging from 2.5 to 12%. This composite system represents a new imaging modality for electric fields that improves on previous designs by using NIR excitation with visible emission, thereby presenting a background-free optical electric field sensor for local electric fields. Additionally, the polymer wrapping enables transfer of the system to a variety of aqueous and organic media.

### B5) Methods

B5a) Nanoparticle synthesis: Hexagonal (β)-phase NaY_{0.8}Yb_{0.18}Er_{0.02}F₄ nanoparticles are synthesized following a procedure based on the literature. All chemicals used were purchased from Sigma-Aldrich, Inc and used as received. Stock solutions of Y(CH₃COOH)₃.xH₂O, Yb(CH₃COOH)₃.4H₂O, Er(CH₃COOH)₃.xH₂O with concentrations of 0.2 M in water are created and stored in a refrigerator. The synthesis begins with the addition of 0.2 mL of the Er stock, 1.8 mL of the Yb stock, and of 8 mL of the Y stock to a 250-mL round-bottomed flask. Oleic acid (15 mL) and octadecene (35 mL) are also added to the flask. The solution is then heated to 150° C. and stirred at temperature for 40 minutes to boil off water and induce the formation of lanthanide-oleate complexes. Afterwards, the reaction is allowed to cool to room temperature while solutions of 1.0 M NaOH and 0.4 M NH₄F in methanol are prepared. Once the reaction has cooled, 5 mL of the NaOH stock and 16.5 mL of the NH₄F stock are added to a 50-mL centrifuge tube and vortexed for 10 seconds before being subsequently injected into the round-bottomed flask. The reaction is then heated to 50° C. and maintained there for 40 minutes to foster the formation of particle nuclei. The temperature is then raised to 100° C. to boil off methanol and any remaining water. After the reaction reaches 100° C., the flask is sealed and vacuum is pulled. The reaction remains at 100° C. under vacuum for 25 minutes. Finally, the reaction is heated further to 315° C. and maintained at temperature in an Ar atmosphere for 90 minutes to ripen the particle nuclei.

B5b) Polymer wrapping: For the polymer wrapping procedure (prior to infiltrating the dye), we follow the literature. Hydrophobic β-NaY_{0.8}Yb_{0.18}Er_{0.02}F₄ UCNPs were dispersed in chloroform with 0.2% (v v⁻¹) oleic acid to 5 μM. 6 mg of poly(maleic anhydride-alt-1-octadecene) amphiphilic copolymer (MW 30-50k, Aldrich) was dissolved to 17 μM in 0.5 mL of acetone and 15 mL of CHCl₃. UCNPs in 100 μL of chloroform were added with stirring, and the solvents were removed under a gentle stream of N₂ over ˜2 hours. UCNP/polymer residue was resuspended in a solution of PEG₃-amine (PurePeg, 10 μmol) in 10 mL of milliQ water. This suspension was sonicated for 60 min, heated in an 80° C. water bath for 60 min, slowly cooled to room temperature, and then sonicated for 30 min. Excess polymer was removed by extensive spin dialysis (Amicon, 100 kDa MWCO), washing 7 times with 15 mL of DI water. The retentate was concentrated to 1.5 mL.

For samples containing dye molecules, DI-2-ANEPEQ (Fisher) dissolved to 0.9 M was added and vortexed for 5 minutes. Excess dye was added until the permeate from spin dialysis contained dye, typically around 1 ml.

B5c) Film preparation and electric field: Films (≈400 nm thick, measured by FIB cross section) were prepared by dropcasting UCNP/polymer solution onto UV ozone plasma cleaned ITO slides. Dropcasting was followed by brief drying at room temperature in vacuum to eliminate residual solvent. A 10 μL droplet of [EMIM] [TFSI] was placed on a polymer/UCNP film and a tungsten electrode was immersed in it. Voltage was applied across the electrodes with the ITO substrate grounded. Unless otherwise noted, any particular voltage was applied for 60-240 s, matched to the optical exposure time.

B5d) Spectroscopic characterization: UC spectra and optical images are acquired under 980 nm illumination from a 980 diode laser (Opto Engine LLC). The spectra are collected using a Princeton Instruments ProEM eXcelon coupled to a Princeton Instruments Acton 2500 spectrometer containing a 500-Blaze 150 groove/mm grating. An 842 SP filter (Semrock BrightLine) is placed at the bottom of the Zeiss brightfield cube to cut off the 980 nm source. The illumination power density is maintained at ˜70 W/cm² incident on the nanoparticle film for 60-240 sec acquisition times per UC spectra.

### B6) Coupling Dyes Without Using Polymer

In addition to the polymer coating method of sections B2-B5 above, we attempted to couple the dyes directly to the nanoparticle surface. To perform these measurements, we used a two phase ligand exchange method, where UCNPs dissolved in cyclohexane were mixed with DI-2-ANEPEQ dissolved in water. After 30 minutes of stirring, the sample was centrifuged (3000 g, 5 min). Based on visual inspection, few dye molecules remain attached to the nanoparticles (the pellet was pale pink indicating slight association between the dye and the nanoparticle). These dye molecules are easily washed off, therefore only a single washing step is performed. Optical characterization of these nanoparticles show quenching of the Er³⁺ green emission, but do not show dye emission. Because these samples have lower dielectric constant than the polymer-wrapped nanoparticles, we can use an air-gap capacitor geometry to apply an electric field across the nanoparticles. When an electric field of ˜130 kV/cm is applied across films of these nanoparticles, an optical response is detected that is similar to that observed for the polymer wrapped particles of sections B2-B5. The same contributions to the optical response as above are present in this case, namely 1) the electronic states of the dye shift, enhancing the dye's absorption of the 525 and 540 nm light and 2) energy back transfer from the dye into the Er³⁺ 640 nm state. We checked the cyclability of the response and find that over 10 electric field cycles the optical response is stable and reproducible.

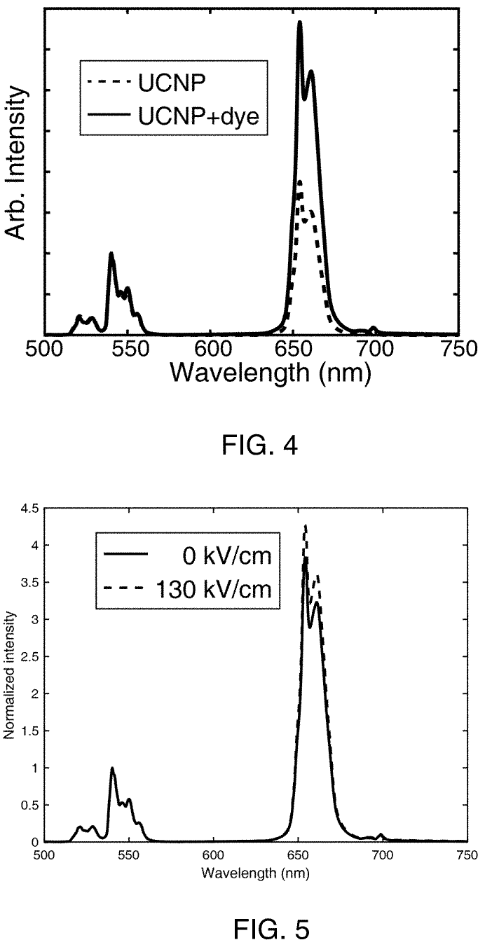

FIG. 4 shows upconversion spectra for UCNPs with (solid line) and without (dashed line) dye. No direct emission from dye molecules is observed, but energy is funneled from the green states to the red states.

FIG. 5 shows upconversion spectra for UCNPs with dye but without polymer. As above, an electric field dependent enhancement of the red emission is observed.

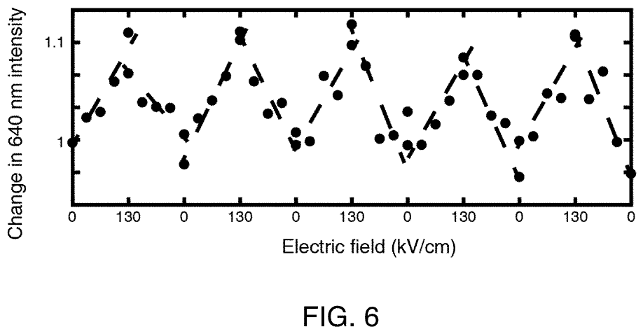

FIG. 6 shows intensity response of red emission as electric field is cycled for the UCNPs with DI-2-ANEPEQ but without polymer. Over 10 cycles, the optical response of the system is approximately the same.

1. A method for sensing electric fields, the method comprising:
providing one or more nanoparticles configured to receive incident radiation at a first wavelength and to provide output radiation at a second wavelength via quantum upconversion, wherein the first and second wavelengths are distinct;
providing one or more dyes having a quantum Stark response to an electric field;
disposing the dyes on or in the nanoparticles such that one or more parameters of the quantum upconversion can be altered by the electric field due to coupling between the dyes and the nanoparticles; and
altering one or more parameters of the quantum upconversion with the electric field.

2. The method of claim 1, wherein the nanoparticles are configured as a core covered by a shell, and wherein the dyes are preferentially disposed in the shell.

3. The method of claim 1, wherein the parameters of the quantum upconversion include intensity at an output wavelength.

4. The method of claim 1, wherein the parameters of the quantum upconversion include spectral intensity in an output wavelength range.

5. The method of claim 1, wherein the nanoparticles are configured to be disposed on or in a biological specimen to provide in vitro electric field sensing with sub-micron spatial resolution.

6. The method of claim 1, wherein the nanoparticles are configured to be disposed on or in a living biological subject to provide in vivo electric field sensing with sub-micron spatial resolution.

7. The method of claim 1, wherein the nanoparticles are configured to be disposed on or in an electrical device or machine to provide in situ electric field sensing with sub-micron spatial resolution.

8. The method of claim 1, wherein the nanoparticles include one or more lanthanide upconverting materials having doping selected from the group consisting of: Er doping, Yb doping, Tm doping, Nd doping and mixtures or combinations thereof.

9. The method of claim 1, wherein the one or more dyes are selected from the group consisting of:
aminonaphthylethenylpyridinium dyes and photoinduced electron transfer dyes.

10. The method of claim 1, wherein the first wavelength is in a range from 0.74 μm to 2.0 μm and wherein the second wavelength is in a range from 380 nm to 740 nm.

11. The method of claim 1, further comprising sensing an electric field at the one or more nanoparticles according to alteration of the one or more parameters of the quantum upconversion.
